(19) United States
(12) Patent Application Publication
(10) Pub. No.: US 2008/0015186 A1
(43) Pub. Date: Jan. 17, 2008

(54) PHARMACEUTICAL COMPOSITIONS CONTAINING IN COMBINATION A CANNABINOID RECEPTOR ANTAGONIST AND AN ANTIPSYCHOTIC

(75) Inventors: Michele ARNONE, Ramonville (FR); Christine RAVINET-TRILLOU, Launaguet (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/854,032

(22) Filed: Sep. 12, 2007

Related U.S. Application Data
(63) Continuation of application No. PCT/FR2006/000532, filed on Mar. 10, 2006.

(30) Foreign Application Priority Data
Mar. 14, 2005 (FR) 0502508

(51) Int. Cl. A61K 31/551; A61K 31/4152; A61K 31/519; A61K 31/454

(52) U.S. Cl. 514/220; 514/326; 514/406; 514/259.41

(57) ABSTRACT

This invention discloses and claims various pharmaceutical compositions containing in combination a pyrazole-based cannabinoid receptor antagonist and an antipsychotic.

This application is a continuation of International application No. PCT/FR2006/000,532, filed Mar. 10, 2006, which is incorporated herein by reference in its entirety; which claims the benefit of priority of French Patent Application No. 0502508, filed Mar. 14, 2005.

The present invention relates to pharmaceutical compositions containing in combination a pyrazole-based cannabinoid receptor antagonist and an antipsychotic.

The term “pyrazole-based cannabinoid CB₁ receptor antagonist” means a compound chosen from N-piperidino-5-(4-chlorophenyl)-1-(2,4-dichlorophenyl)-4-methylpyrazole-3-carboxamide, the international nonproprietary name of which is rimonabant, described in European patent 656 354, and N-piperidino-5-(4-bromophenyl)-1-(2,4-dichlorophenyl)-4-ethylpyrazole-3-carboxamide, described in European patent 1 150 961; the pharmaceutically acceptable salts or solvates of each of these compounds are also included.

The term “antipsychotic” means compounds such as: risperidone, olanzapine, clozapine, sertindole, zotipine, seroquel; the pharmaceutically acceptable salts or solvates of each of these compounds are also included.

The pharmaceutical composition according to the present invention is useful for preventing and treating excess weight, obesity and metabolic disorders such as fat and carbohydrate metabolism disorders, associated with schizophrenia and with its treatment with antipsychotics.

The pharmaceutical compositions according to the present invention contain an effective dose of a pyrazole-based cannabinoid CB₁ receptor antagonist, and an antipsychotic, and also at least one pharmaceutically acceptable excipient.

Said excipients are chosen, according to the pharmaceutical form and the desired mode of administration, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active principle may be administered in a unit administration form, as a mixture with standard pharmaceutical excipients, to man and animals for preventing or treating the above disorders or diseases.

The appropriate unit administration forms include oral forms such as tablets, soft or hard gel capsules, powders, granules and oral solutions or suspensions, sublingual, buccal, intratracheal, intraocular, intranasal or inhalation administration forms, topical, transdermal, subcutaneous, intramuscular or intravenous administration forms, rectal administration forms and implants. For topical application, the compounds according to the invention may be used in creams, gels, ointments or lotions.

Most particularly, a subject of the present invention is a pharmaceutical composition containing in combination a pyrazole-based cannabinoid CB₁ receptor antagonist chosen from rimonabant and N-piperidino-5-(4-bromophenyl)-1-(2,4-dichlorophenyl)-4-ethylpyrazole-3-carboxamide or a pharmaceutically acceptable salt thereof or a solvate thereof and an antipsychotic, and at least one pharmaceutically acceptable excipient. A subject of the present invention is particularly a pharmaceutical composition containing in combination rimonabant or N-piperidino-5-(4-bromophenyl)-1-(2,4-dichlorophenyl)-4-ethylpyrazole-3-carboxamide or a pharmaceutically acceptable salt thereof or a solvate thereof and an antipsychotic chosen from risperidone, olanzapine, clozapine, sertindole, zotipine and seroquel, or a pharmaceutically acceptable salt thereof or a solvate thereof.

More particularly, a subject of the present invention is a pharmaceutical composition containing in combination rimonabant and risperidone and at least one pharmaceutically acceptable excipient.

According to another aspect of the invention, the pyrazole-based cannabinoid receptor antagonist and the combined antipsychotic may be administered simultaneously, separately or sequentially.

The term “simultaneous use” means the administration of the compounds of the composition according to the invention included in the same pharmaceutical form.

The term “separate use” means the administration, at the same time, of the two compounds of the composition according to the invention, each included in a separate pharmaceutical form.

The term “sequential use” means the successive administration of the first compound of the composition according to the invention, included in one pharmaceutical form, followed by the second compound of the composition according to the invention, included in a separate pharmaceutical form.

In the case of this “sequential use”, the interval between the administration of the first compound of the composition according to the invention and the administration of the second compound of the same composition according to the invention generally does not exceed 24 hours; it may be longer if one or other of the compounds is presented in a pharmaceutical form allowing, for example, a weekly administration.

The pharmaceutical forms containing either only one of the constituent compounds of the composition according to the invention, or the combination of the two compounds that may be used in the various types of use described above, may be suitable, for example, for oral, nasal, parenteral or transdermal administration.

Also, in the case of a “separate use” and of a “sequential use”, two separate pharmaceutical forms may be intended for the same administration route or for a different administration route (oral and transdermal, or oral and nasal, or parenteral and transdermal, etc.).

The invention thus also relates to a kit containing a pyrazole-based cannabinoid CB₁ receptor antagonist and an antipsychotic, in which said pyrazole-based cannabinoid CB₁ receptor antagonist and said antipsychotic are in separate compartments and in identical or different packaging, and are intended to be administered simultaneously, separately or sequentially.

### EXAMPLE 1

### Effect of Rimonabant on the Weight Gain Induced by Treatment with Risperidone

Female Wistar rats, fed with a fat-rich feed for the three days preceding the treatment and during the treatment, are used.

Groups of 15 animals receive each day:
group 1: risperidone (0.3 mg/kg, i.p.) and the vehicle (p.o.);
group 2: risperidone (0.3 mg/kg, i.p.) and rimonabant (1 mg/kg, p.o.);
group 3: the two vehicles (i.p. and p.o.);

On the ninth day of the treatment, the cumulative weight gain over the nine days of treatment and the feed consumption on day 9 are measured.

TABLE 1
Cumulative weight gain over the nine days of treatment

Number of rats/group (%) with a
weight gain

| Groups | Weight gain | <20 g | >20 g |
|---|---|---|---|
| Group 1 | 23.5 g ± 1.7 * | 20% | 80% |
| Group 2 | 11.4 g ± 2.2 *$ | 93% | 7% |
| Group 3 | 17.2 g ± 2.1 | 67% | 33% |

TABLE 2
Mean feed intake on the ninth day

| Animals | Feed intake |
|---|---|
| Group 1 | 15.0 g ± 0.7 * |
| Group 2 | 12.7 g ± 0.5 $ |
| Group 3 | 13.2 g ± 0.5 |

* p < 0.05 versus group 3
$ p > 0.05 between group 1 and group 2.

It is observed that the combination of rimonabant at 1 mg/kg and risperidone at 0.3 mg/kg prevents the increase in weight induced by the treatment with risperidone.

What is claimed is:

1. A pharmaceutical composition comprising, in combination, at least one active ingredient chosen from a pyrazole-based cannabinoid CB₁ receptor antagonist and pharmaceutically acceptable salts thereof and at least one second active ingredient chosen from an antipsychotic agent, further in combination with at least one pharmaceutically acceptable excipient.

2. The composition according to claim 1, wherein the at least one active ingredient is chosen from rimonabant, N-piperidino-5-(4-bromophenyl)-1-(2,4-dichlorophenyl)-4-ethylpyrazole-3-carboxamide and pharmaceutically acceptable salts thereof.

3. The composition according to claim 2, wherein the at least one active ingredient is chosen from rimonabant and pharmaceutically acceptable salts thereof.

4. The composition according to claim 2, wherein the at least one active ingredient is chosen from N-piperidino-5-(4-bromophenyl)-1-(2,4-dichlorophenyl)-4-ethylpyrazole-3-carboxamide and pharmaceutically acceptable salts thereof.

5. The composition according to claim 1, wherein the antipsychotic is chosen from risperidone, olanzapine, clozapine, sertindole, zotipine and seroquel and pharmaceutically acceptable salts thereof or solvates thereof.

6. The composition according to claim 1, wherein the antipsychotic is chosen from risperidone and pharmaceutically acceptable salts thereof or solvates thereof.

7. The composition according to claim 1, wherein the antipsychotic is chosen from olanzapine and pharmaceutically acceptable salts thereof or solvates thereof.

8. The composition according to claim 1, wherein the antipsychotic is chosen from clozapine and pharmaceutically acceptable salts thereof or solvates thereof.

9. The composition according to claim 1, wherein the antipsychotic is chosen from sertindole and pharmaceutically acceptable salts thereof or solvates thereof.

10. The composition according to claim 1, wherein the antipsychotic is chosen from zotipine and pharmaceutically acceptable salts thereof or solvates thereof.

11. The composition according to claim 1, wherein the antipsychotic is chosen from seroquel and pharmaceutically acceptable salts thereof or solvates thereof.

12. The composition according to claim 1, containing in combination rimonabant or a pharmaceutically acceptable salt thereof or a solvate thereof and risperidone or a pharmaceutically acceptable salt thereof or a solvate thereof.

13. A method of controlling weight gain in a patient comprising administering to the patient a therapeutically effective amount of a combination of at least one active ingredient chosen from a pyrazole-based cannabinoid CB₁ receptor antagonist and pharmaceutically acceptable salts thereof and at least one second active ingredient chosen from an antipsychotic agent and optionally in combination with one or more pharmaceutically acceptable excipients.

14. The method according to claim 12, wherein the at least one active ingredient is chosen from rimonabant, N-piperidino-5-(4-bromophenyl)-1-(2,4-dichlorophenyl)-4-ethylpyrazole-3-carboxamide and pharmaceutically acceptable salts thereof.

15. The method according to claim 14, wherein the at least one active ingredient is chosen from rimonabant and pharmaceutically acceptable salts thereof.

16. The method according to claim 14, wherein the at least one active ingredient is chosen from N-piperidino-5-(4-bromophenyl)-1-(2,4-dichlorophenyl)-4-ethylpyrazole-3-carboxamide and pharmaceutically acceptable salts thereof.

17. The method according to claim 12, wherein the antipsychotic is chosen from risperidone, olanzapine, clozapine, sertindole, zotipine and seroquel and pharmaceutically acceptable salts thereof or solvates thereof.

18. The method according to claim 12, wherein the antipsychotic is chosen from risperidone and pharmaceutically acceptable salts thereof or solvates thereof.

19. The method according to claim 12, wherein the antipsychotic is chosen from olanzapine and pharmaceutically acceptable salts thereof or solvates thereof.

20. The method according to claim 12, wherein the antipsychotic is chosen from clozapine and pharmaceutically acceptable salts thereof or solvates thereof.

21. The method according to claim 12, wherein the antipsychotic is chosen from sertindole and pharmaceutically acceptable salts thereof or solvates thereof.

22. The method according to claim 12, wherein the antipsychotic is chosen from zotipine and pharmaceutically acceptable salts thereof or solvates thereof.

23. The method according to claim 12, wherein the antipsychotic is chosen from seroquel and pharmaceutically acceptable salts thereof or solvates thereof.

24. The method according to claim 12, the combination is rimonabant or a pharmaceutically acceptable salt thereof or a solvate thereof and risperidone or a pharmaceutically acceptable salt thereof or a solvate thereof.

25. A kit comprising a pyrazole-based cannabinoid CB₁ receptor antagonist chosen from rimonabant and N-piperidino-5-(4-bromophenyl)-1-(2,4-dichlorophenyl)-4-ethylpyrazole-3-carboxamide, or a pharmaceutically acceptable salt thereof or a solvate thereof, and an antipsychotic chosen from risperidone, olanzapine, clozapine, sertindole, zotipine and seroquel, or a pharmaceutically acceptable salt thereof or a solvate thereof, in which the cannabinoid CB₁ receptor antagonist and the antipsychotic are in separate compartments and in identical or different packaging, and are intended to be administered simultaneously, separately or sequentially.
